(19) United States
(12) Patent Application Publication
(10) Pub. No.: US 2009/0260627 A1
(43) Pub. Date: Oct. 22, 2009

(54) LIQUID ANESTHETIC CONTAINER AND DELIVERY SYSTEM

(76) Inventors: Michael CUZYDLO, Orchard Park, NY (US); Shannon X. Qin, Fort Erie (CA)

Correspondence Address: REED SMITH LLP, P.O. BOX 488, PITTSBURGH, PA, 15230-0488, US

(21) Appl. No.: 12/406,884

(22) Filed: Mar. 18, 2009

Related U.S. Application Data
(60) Provisional application No. 61/037,606, filed on Mar. 18, 2008.

(51) Int. Cl. A61M 16/01; B65D 41/04

(52) U.S. Cl. 128/203.21; 220/288

(57) ABSTRACT

A device for containing a volatile anesthetic and an anesthetic agent delivery system. The device may include a bottle closure, which may be attached to an anesthetic bottle until it is time for the anesthetic to be transferred to a vaporizer. A membrane may be used to seal the opening in order to prevent the anesthetic from leaving the reservoir until it is desired to fill the vaporizer. In order to protect the membrane a cap and a cap support may be placed on the reservoir so as to cover the membrane. The container may further include a connecting surface on the cap support or the reservoir, the connecting surface being connectable to a vaporizer. The system includes a vaporizer having a side slotted tube needle that is able to pierce the membrane and through which the liquid anesthetic agent and/or vapor may pass from the reservoir to the vaporizer. A method for transferring a liquid anesthetic from a container to a vaporizer. The method includes connecting a container which holds a liquid anesthetic to a vaporizer, and piercing a membrane of the container with a slotted needle of the vaporizer.

### CROSS-REFERENCE TO RELATED APPLICATION

This application claims the benefit under 35 U.S.C. § 119(e) of the earlier filing date of U.S. Provisional Application Ser. No. 61/037,606 filed on Mar. 18, 2008, the entire disclosure of which is hereby incorporated by reference herein as if being set forth in its entirety.

### FIELD OF THE INVENTION

This application discloses an invention which is related, generally and in various embodiments, to a container for supplying a liquid anesthetic to a vaporizer, an anesthetic delivery system, and a method for transferring a liquid anesthetic from a container to a vaporizer.

### BACKGROUND OF THE INVENTION

Liquid anesthetics are often packaged in glass bottles and shipped to a location where they may be used to anesthetize a patient undergoing a medical or dental procedure. Such anesthetics may also be used to induce analgesia or sedation in a patient who is undergoing a medical or dental procedure. In order to administer the anesthetic, the contents of the glass bottle are placed in a vaporizer. The vaporizer may be used to vaporize the anesthetic, and provide the vaporized anesthetic in a desired amount to the patient.

Inhalable anesthetics are typically volatile substances with relatively low boiling points and high vapor pressures. Preferably, the anesthetic should be used in a way which will ensure that there is little or no release to the atmosphere at all stages of handling. In order to transfer the liquid anesthetic to the vaporizer, the bottle must be opened. Since it is unwise to expose medical personnel performing the procedure to the anesthetic, and since anesthetics are expensive, devices have been developed to minimize or eliminate the release of the anesthetic from the bottle to the environment surrounding the vaporizer.

### SUMMARY OF THE INVENTION

In one general respect, this application discloses a container for supplying a liquid anesthetic to a vaporizer. According to various embodiments, the container includes a reservoir, a membrane, a cap support, and a cap. The reservoir is adapted for holding the liquid anesthetic. The membrane covers an open end of the reservoir. The cap support is connected to the reservoir. The cap is removably connected to the cap support.

In another general respect, this application discloses an anesthetic delivery system. According to various embodiments, the system includes a container for supplying a liquid anesthetic, and a vaporizer configured to receive the container. The container includes a reservoir, a membrane, a cap support, and a cap. The reservoir is adapted for holding a liquid anesthetic. The membrane covers an opening of the reservoir. The cap support is connected to the reservoir. The cap is removably connected to the cap support. The vaporizer includes a slotted tube. The slotted tube is configured to pierce the membrane.

In yet another general respect, this application discloses a method for transferring a liquid anesthetic from a container to a vaporizer. According to various embodiments, the method includes connecting a container which holds a liquid anesthetic to a vaporizer, and piercing a membrane of the container with a slotted needle of the vaporizer.

### BRIEF DESCRIPTION OF THE DRAWINGS

FIG. 1A illustrates a device according to the invention.

FIG. 1B illustrates an exploded view of the device of FIG. 1A.

FIG. 2A illustrates another device according to the invention.

FIG. 2B illustrates an exploded view of the device of FIG. 2A.

FIG. 3 illustrates another device according to the invention.

FIG. 4 is an exploded and partial cross sectional view of a delivery system according to the invention illustrated in FIG. 1A showing the position and relationship of the anesthetic bottle and a receiving station of a vaporizer.

FIG. 5 is an exploded view of another delivery system according to the invention illustrated in FIG. 2A showing the position and relationship of the anesthetic bottle and a receiving station of a vaporizer.

FIG. 6 is an exploded view of yet another delivery system according to the invention illustrated in FIG. 3 showing the position and relationship of the anesthetic bottle and a receiving station of a vaporizer.

FIG. 7A illustrates a perspective view of a side slotted tube that may be used in a system according to the invention.

FIG. 7B illustrates a side view of the side slotted tube illustrated in FIG. 7A.

FIG. 7C illustrates an end view of the side slotted tube illustrated in FIG. 7A.

FIG. 7D illustrates a cross-sectional view of the side slotted tube illustrated in FIG. 7A.

FIG. 8A illustrates a perspective view of another embodiment side slotted tube that may be used in a system according to the invention.

FIG. 8B illustrates a side view of the side slotted tube illustrated in FIG. 8A.

FIG. 8C illustrates an end view of the side slotted tube illustrated in FIG. 8A.

FIG. 8D illustrates a cross-sectional view of the side slotted tube illustrated in FIG. 8A.

### DETAILED DESCRIPTION OF THE INVENTION

The accompanying drawings are intended to provide further understanding of the invention and are incorporated in and constitute a part of the description of the invention. The drawings illustrate an embodiment of the invention and together with the description illustrate principles of the invention.

The drawings should not be taken as implying any necessary limitation on the essential scope of invention.

The drawings are given by way of non-limitative example to explain the nature of the invention.

For a more complete understanding of the instant invention reference is now made to the following description taken in conjunction with accompanying drawings.

The various features of novelty which characterize the invention are pointed out specifically in the claims which are a part of this description. For a better understanding of the invention, reference should be made to the drawings and descriptive matter in which there are illustrated and described preferred embodiments of invention.

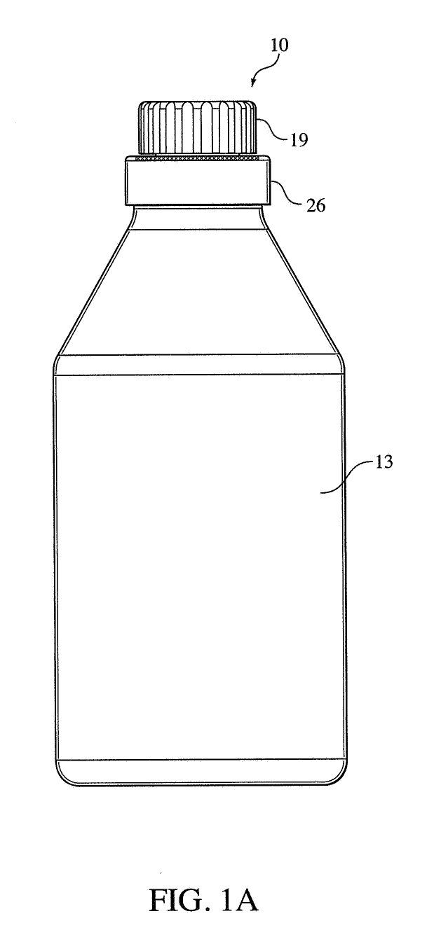

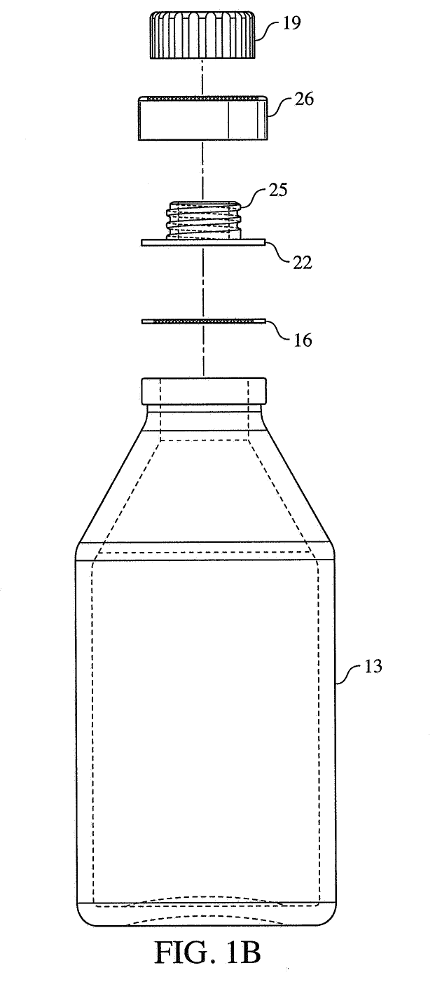

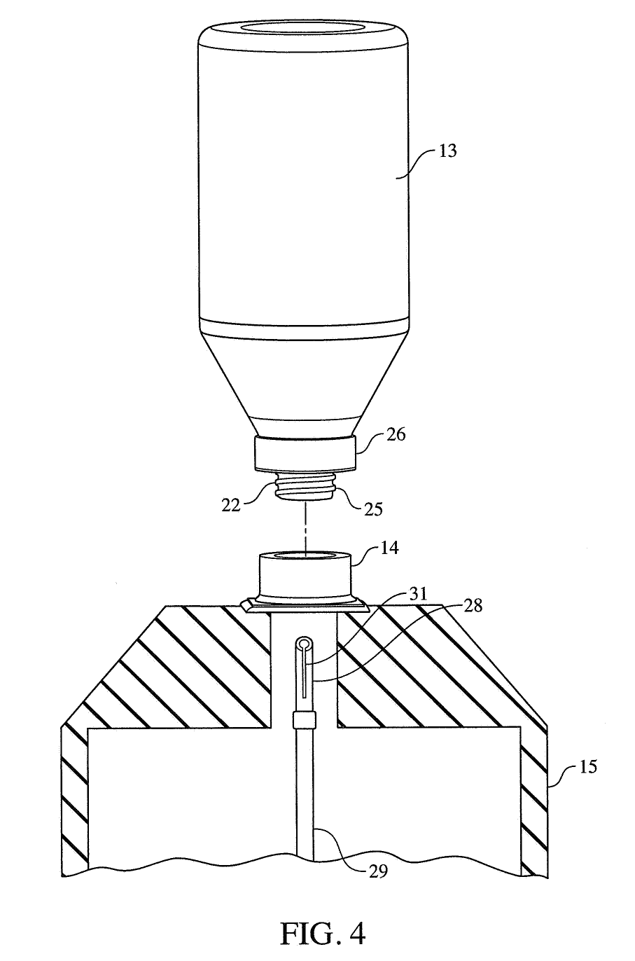

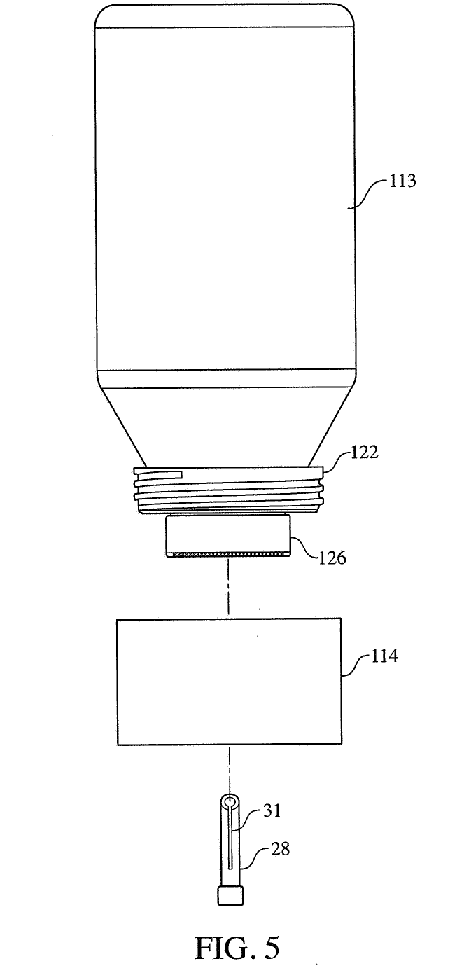

Referring now to the drawings, wherein like numerals designate identical or corresponding parts throughout the referred views, in FIGS. 1A and 1B, there is shown a device for containing a volatile anesthetic according to various embodiments of the invention. Such a device may include a bottle closure 10, which may be attached to an anesthetic bottle (a “reservoir 13”) in order to keep the anesthetic in the reservoir 13 until it is time for the anesthetic to be transferred to a vaporizer. A receiving station 14 of the vaporizer 15 is shown in FIG. 4. In one embodiment of the invention, there is a reservoir 13, which holds the liquid anesthetic agent. The reservoir 13 may have an opening through which the anesthetic may flow to the vaporizer 15. A membrane 16 may be used to seal the opening in order to prevent the anesthetic from leaving the reservoir 13 until it is desired to fill the vaporizer 15. The membrane 16 may be made of rubbery material such as FDA grade ethylene propylene diene M-class “EPDM” rubber, which provides a seal between the anesthetic agent and the environment.

In order to protect the membrane 16, a cap 19 and a cap support 22 may be placed on the reservoir 13 so as to cover the membrane 16. The cap 19 may be made of a durable material that is difficult to puncture. The cap support 22 may have a connection surface 25 that may be used (1) to hold the cap 19 relative to the membrane 16, or (2) to hold the reservoir 13 relative to the vaporizer 15, and may also be used to form a seal between the reservoir 13 and the vaporizer, or (3) may be used to accomplish item (1) and then to accomplish item (2). It should be noted that the connecting surface 25 need not be on the cap support 22. For example, the connecting surface 25 may be part of the reservoir 13 and also may be internally or externally threaded.

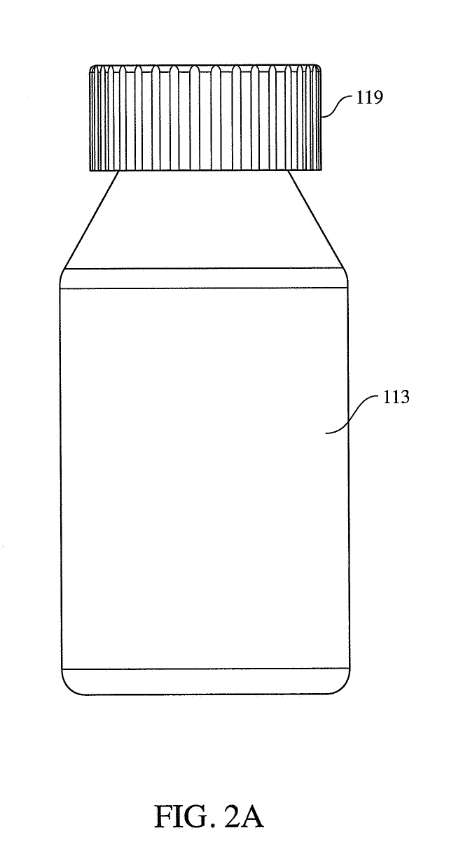

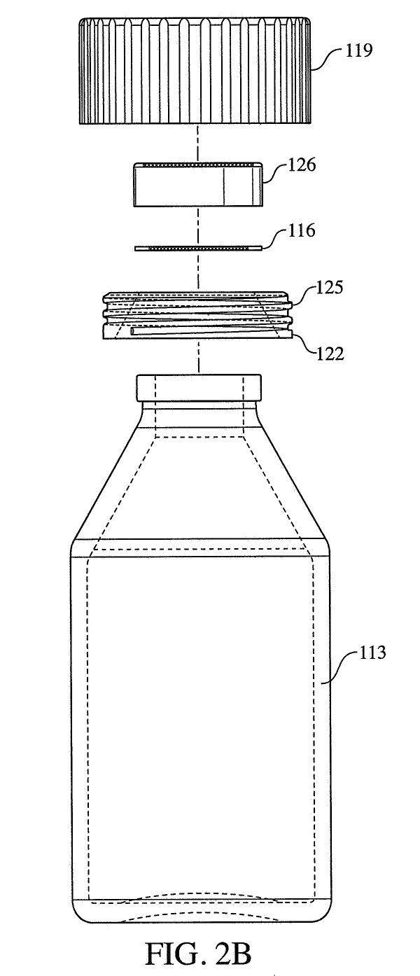

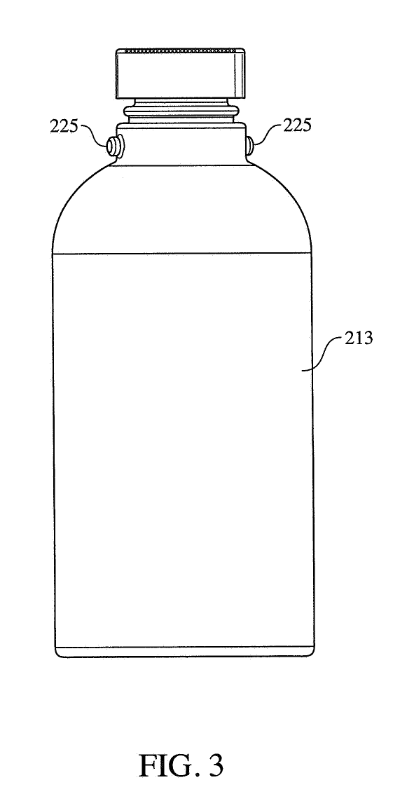

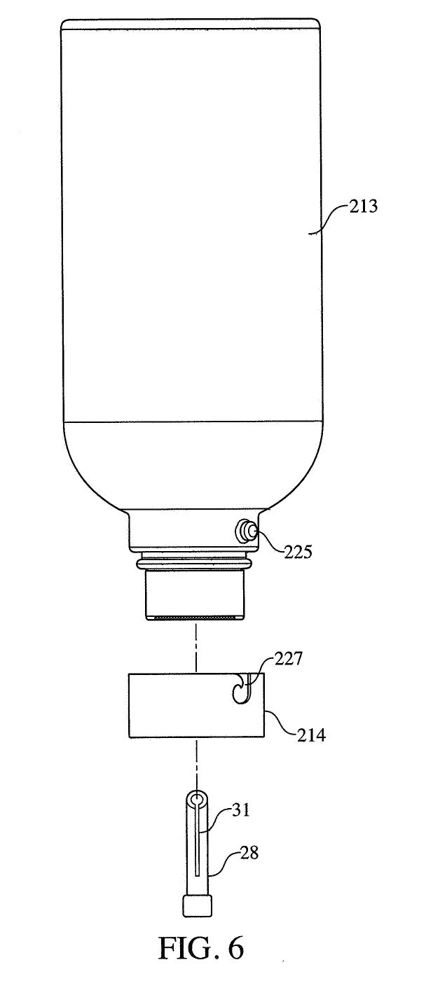

A threaded connection surface 25 is shown in the embodiment of FIGS. 1A, 1B. Another threaded connection surface 125 is shown in the embodiment of FIGS. 2A, 2B in which the cap support 122 is receivable on the outside of a neck of the reservoir 113 to support the cap 119. If a threaded connection surface is used, the vaporizer receiving station 14, 114 may have threads which engage the connection surface 25, 125. Alternatively, the connection surface may have one or more male bayonet extensions 225 that are connectable with slotted openings 227 (see FIGS. 3 and 6) in the vaporizer receiving station 214. The slotted openings 227 are provided to receive, and then upon turning the reservoir 213, capture the male bayonet extensions 225.

In the embodiment of FIGS. 1A, 1B, liquid anesthetic agent is placed in the reservoir 13 and the membrane 16 is placed on the reservoir 13 so as to cover the opening of the reservoir 13. Then, the cap support 22 may be placed on the reservoir 13 and the cap support 22 may be crimped to the reservoir 13 by a crimp ring 26 in order to prevent the membrane 16 from becoming detached from the reservoir 13. The cap support 22 may be crimped so as to engage a neck of the reservoir 13. In the embodiment of FIGS. 2A, 2B, the membrane 116 may be crimped in place by crimp ring 126. FIG. 3 shows an embodiment of the invention in which the cap support 222 is not crimped, but nevertheless the cap support 222 is held to a neck of the reservoir 213.

Referring again to the embodiment of FIGS. 1A, 1B and 4, by way of example only, when it is desired to remove the liquid anesthetic from the reservoir 13, and place the anesthetic in the vaporizer, the cap 19 may be removed, thereby exposing the membrane 16. The reservoir 13 may be inverted so that the force of gravity places the liquid anesthetic against the membrane 16. The membrane 16 and the cap support 22 may then be inserted into a receiving station of the vaporizer 15 until the connecting surface 25 on the cap support 22 can be engaged with the vaporizer. Once the reservoir 13 is engaged with the vaporizer, the membrane 16 may be punctured and the liquid anesthetic may be allowed to flow via the force of gravity from the reservoir 13 to the vaporizer sump.

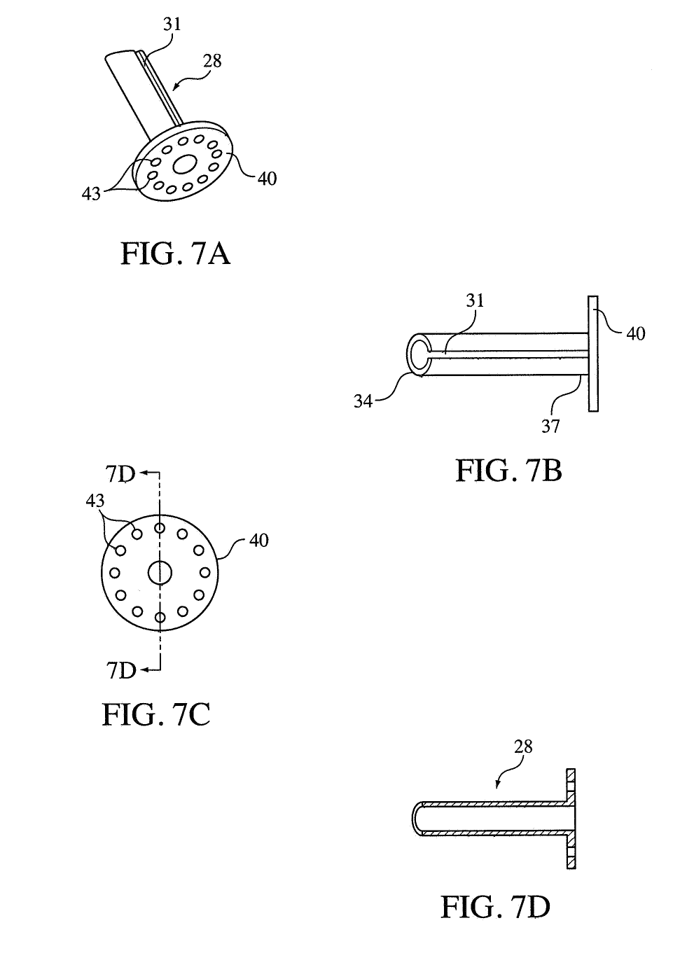

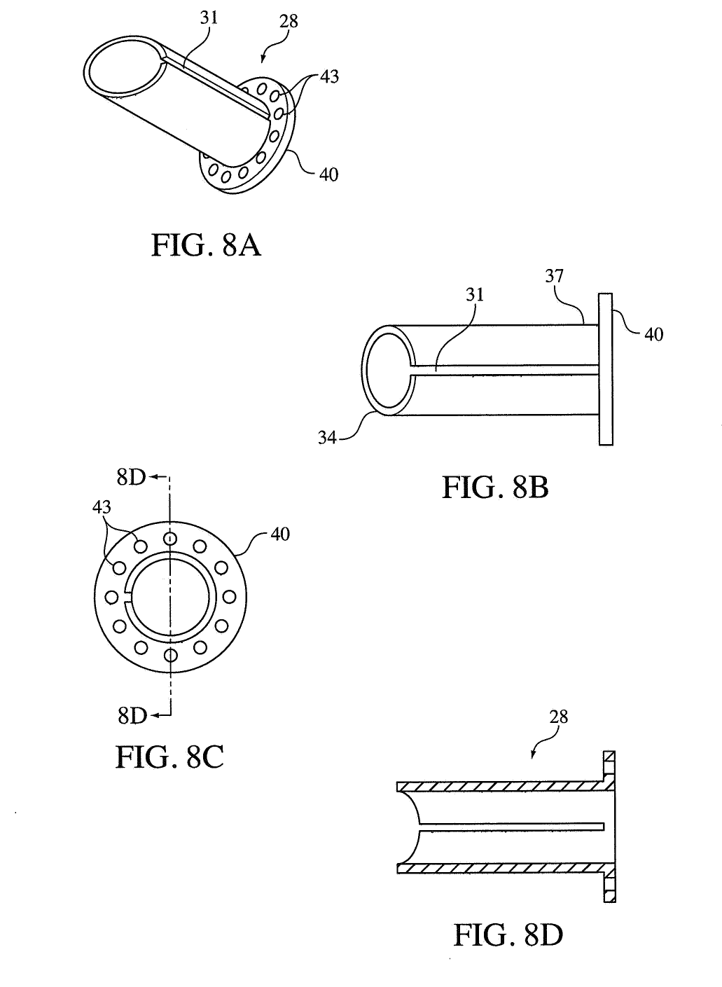

To puncture the membrane 16, the vaporizer may be equipped with a side slotted needle or tube 28, the details of which are illustrated in FIGS. 7A-8D. FIGS. 8A-8D depict an embodiment of a side-slotted tube having a larger diameter opening than the embodiment of the side slotted tube illustrated in FIGS. 7A-7D. The side slotted tube 28 may be moved through the membrane by, for example, a mechanical actuator 29 in order to puncture the membrane 16. Alternatively, the reservoir 13 may be moved so as to allow the side slotted tube 28 to puncture the membrane 16. Liquid anesthetic may flow through the tube 28 from the reservoir 13 to the vaporizer.

The side slotted tube 28 may be a cylindrical tube with one or more longitudinally extending slots 31 in the wall of the tube 28. The slot 31 may be positioned so that when the tube 28 extends through the membrane 16, the slot 31 extends from one side of the membrane 16 to the other side of the membrane 16. In this fashion, liquid anesthetic may enter the tube 28 through an end portion of the tube 28 and/or through the slot 31. When the level of liquid anesthetic in the reservoir 13 drops below the end of the tube 28, the liquid anesthetic will enter the tube 28 only through the slot 31. Since the slot 31 extends from one side of the membrane 16 to the other, all of the anesthetic in the reservoir 13 is allowed to drain from the reservoir 13 to the vaporizer.

The side slotted tube 28 includes a piercing end 34 for puncturing the membrane 16 and a base end 37 opposite thereto. The side slotted tube 28 further includes a radially extending flange 40 disposed at its base end 37. Flange 40 includes a plurality of radially spaced through holes 43 extending from one side of flange 40 to the other. Through holes 43 allow drainage in both directions of fluid and vapor.

When it is desired to remove the reservoir 13 from the vaporizer 15, the tube 28 is removed from the membrane 16. If the membrane 16 is made from EPDM rubber, the hole through which the tube 28 extends may be re-sealed by the material's “self-curing” property. In this manner, the reservoir 13 may be re-sealed after the liquid anesthetic agent is transferred to the vaporizer.

Various embodiments provide for an anesthetic agent to be sealed and stored in a container under controlled conditions. The agent is then able to remain in the container, and not be exposed to the atmosphere prior to administration to a patient.

It is to be understood that the invention may assume various alternative orientations and step sequences, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. A container for supplying a liquid anesthetic to a vaporizer, the container comprising:
a reservoir adapted for holding the liquid anesthetic;
a membrane which covers an open end of the reservoir;
a cap support connected to the reservoir; and
a cap removably connected to the cap support.

2. The container of claim 1, further comprising a connecting surface configured for connection to the vaporizer.

3. The container of claim 2, wherein the connecting surface is defined by one of the following:
the cap support; and
the reservoir.

4. The container of claim 2, wherein the connecting surface defines at least one of the following:
an internal thread;
an external thread; and
a male bayonet extension.

5. The container of claim 1, further comprising a crimp ring which surrounds the cap support.

6. An anesthetic delivery system comprising:
a container for supplying a liquid anesthetic, the container comprising:
a reservoir adapted for holding the liquid anesthetic;
a membrane which covers an open end of the reservoir;
a cap support connected to the reservoir; and
a cap removably connected to the cap support; and
a vaporizer configured to receive the container, wherein the vaporizer comprises:
a slotted tube, wherein the slotted tube is configured to pierce the membrane.

7. The system of claim 6, wherein the container further comprises a connecting surface configured for connection to the vaporizer.

8. The system of claim 7, wherein the connecting surface is defined by one of the following:
the cap support; and
the reservoir.

9. The system of claim 7, wherein the connecting surface defines at least one of the following:
an internal thread;
an external thread; and
a male bayonet extension.

10. The system of claim 6, wherein the slotted tube defines at least one longitudinal slot through a wall of the tube.

11. The system of claim 6, further comprising an actuator connected to the slotted tube.

12. The system of claim 6, further comprising a flange connected to the slotted tube.

13. The system of claim 12, wherein the flange defines a plurality of through holes.

14. A method for transferring a liquid anesthetic from a container to a vaporizer, the method comprising:
connecting a container which holds a liquid anesthetic to a vaporizer; and
piercing a membrane of the container with a slotted needle of the vaporizer.

15. The method of claim 14, wherein connecting the container comprises threadedly connecting the container to the vaporizer.

16. The method of claim 14, wherein connecting the container comprises engaging a male bayonet extension of the container with a slotted opening of the vaporizer.

17. The method of claim 14, wherein piercing the membrane comprises moving the slotted tube from a first position to a second position.

18. The method of claim 17, wherein moving the slotted tube comprises moving the slotted tube with an actuator of the vaporizer.

19. The method of claim 17, further comprising moving the slotted tube from the second position to the first position.

20. The method of claim 14, further comprising removing the slotted tube from contact with the membrane.
